(19) United States
(12) Patent Application Publication
(10) Pub. No.: US 2011/0171683 A1
(43) Pub. Date: Jul. 14, 2011

(54) METHODS AND COMPOSITIONS FOR ENUMERATING ANTIBIOTIC-RESISTANT MICROORGANISMS

(76) Inventors: Patrick A. Mach, Shorewood, MN (US); Michelle L. Rosauer, North St. Paul, MN (US)

(21) Appl. No.: 13/059,303

(22) PCT Filed: Aug. 19, 2009
(86) PCT No.: PCT/US09/54240

§ 371 Date: Mar. 8, 2011

Related U.S. Application Data
(60) Provisional application No. 61/090,761, filed on Aug. 21, 2008.

(51) Int. Cl. C12Q 1/14; C12Q 1/04; C12Q 1/06

(52) U.S. Cl. 435/36; 435/34; 435/39

(57) ABSTRACT

A thin film culture device for detection of antibiotic-resistant microorganisms is provided. The device can include indicators to differentiate staphylococcal from non-staphylococcal microorganisms. Methods of use include detecting or enumerating antibiotic-resistant microorganisms. The methods further include obtaining a differential count of staphylococcal and non-staphylococcal microorganisms.

### CROSS REFERENCE TO RELATED APPLICATIONS

This application claims priority to U.S. Patent Application No. 61/090,761, filed Aug. 21, 2008, which is hereby incorporated herein by reference in its entirety.

### BACKGROUND

The coagulase-positive species Staphylococcus aureus is well documented as a human opportunistic pathogen (Murray et al. Eds, 1999, Manual of Clinical Microbiology, 7th Ed., ASM Press, Washington, D.C.). Nosocomial infections caused by S. aureus are a major cause of morbidity and mortality. Some of the most common infections caused by S. aureus involve the skin, and they include furuncles or boils, cellulitis, impetigo, and postoperative and chronic wound infections at various sites. Some of the more serious infections produced by S. aureus are bacteremia, pneumonia, osteomyelitis, acute endocarditis, myocarditis, pericarditis, cerebritis, meningitis, scalded skin syndrome, and various abscesses.

Food poisoning mediated by staphylococcal enterotoxins is another important syndrome associated with S. aureus. Toxic shock syndrome, a community-acquired disease, has also been attributed to infection or colonization with toxigenic S. aureus. Methicillin-resistant S. aureus (MRSA) emerged in the 1980s as a major clinical and epidemiologic problem in hospitals (Oliveira et al., 2002, Lancet Infect Dis. 2:180-189). MRSA are resistant to all β-lactams; including penicillins, cephalosporins, carbapenems, and monobactams; which are the most commonly used antibiotics to cure S. aureus infections. MRSA infections can only be treated with more toxic and more costly antibiotics, which are normally used as the last line of defense. Since MRSA can spread easily from patient to patient via healthcare personnel, hospitals around the world are confronted with the problem to control MRSA. Consequently, there is a need to develop rapid and simple screening or diagnostic tests for detection and/or identification of MRSA to reduce its dissemination and improve the diagnosis and treatment of infected patients.

Methicillin resistance in S. aureus is unique in that it is due to acquisition of DNA from other coagulase negative staphylococci (CNS), coding for a supernumerary β-lactam-resistant penicillin-binding protein (PBP), which takes over the biosynthetic functions of the normal PBPs when the cell is exposed to β-lactam antibiotics. S. aureus normally contains four PBPs, of which PBPs 1, 2 and 3 are essential. The low-affinity PBP in MRSA, termed PBP 2a (or PBP2′), is encoded by the chromosomal mecA gene and functions as a β-lactam-resistant transpeptidase. The mecA gene is absent from methicillin-sensitive S. aureus but is widely distributed among other species of staphylococci and is highly conserved (Ubukata et al., 1990, Antimicrob. Agents Chemother. 34: 170-172).

Methods to detect and identify MRSA based on the detection of the mecA gene and S. aureus-specific chromosomal sequences have been described. (Saito et al., 1995, J. Clin. Microbiol. 33:2498-2500; Ubukata et al., 1992, J. Clin. Microbiol. 30:1728-1733; Murakami et al., 1991, J. Clin. Microbiol. 29:2240-2244; Hiramatsu et al., 1992, Microbiol. Immunol. 36:445-453). However, because the mecA gene is widely distributed in both S. aureus and coagulase-negative staphylococci, these methods are not always capable of discriminating MRSA from methicillin-resistant coagulase-negative staphylococci (MRCNS, see, Suzuki et al., 1992, Antimicrob. Agents. Chemother. 36:429-434). To address this problem, Hiramatsu et al. developed a PCR-based assay specific for MRSA that utilizes primers that hybridize to the right extremities of the 3 types of Staphylococcal Chromosomal Cassette mec DNAs (SCCmec DNAs) in combination with primers specific to the S. aureus chromosome, which corresponds to the nucleotide sequence on the right side of the SCCmec integration site. (U.S. Pat. No. 6,156,507). Nucleotide sequences surrounding the SCCmec integration site in other staphylococcal species (e.g., S. epidermidis and S. haemolyticus) are different from those found in S. aureus; therefore, this PCR assay is specific for the detection of MRSA.

Genetic assays to detect MRSA and to differentiate MRSA from MRCNS are relatively expensive. Additionally, such tests require sophisticated equipment and skilled laboratory personnel to conduct them.

### SUMMARY

In view of the current tests, which require sophisticated equipment, labile culture media and/or highly-skilled laboratory personnel, there is a need for a simpler, culture-based test to detect and differentiate MRSA and/or MRCNS microorganisms in a sample. Furthermore, current tests for MRSA and MRCNS microorganisms are either qualitative or semi-quantitative. The present disclosure relates to simple articles and methods for detecting and differentiating antibiotic-resistant microorganisms in a sample. Advantageously, certain embodiments of the present disclosure provide for quantitative enumeration of antibiotic-resistant microorganisms. The inventive methods also provide for the differential enumeration of MRSA and MRCNS in a sample containing both microorganisms.

In one aspect, the present disclosure provides an article for detecting or enumerating antibiotic-resistant microorganisms. The article can comprise an effective amount of a β-lactam antibiotic to select for the growth of antibiotic-resistant staphylococcal microorganisms, with the proviso that the β-lactam antibiotic is not aztreonam. The article can further comprise a nutrient medium, an indicator system to indicate the presence of microorganisms, and a dry, rehydratable thin film culture device comprising a gelling agent.

In another aspect, the present disclosure provides an article for differentially enumerating antibiotic-resistant microorganisms. The article can comprise an effective amount of a β-lactam antibiotic to select for the growth of antibiotic-resistant staphylococcal microorganisms, with the proviso that the β-lactam antibiotic is not aztreonam. The article can further comprise a nutrient medium, a first indicator system to indicate the presence of microorganisms, a second indicator system to indicate the presence of Staphylococcus aureus, and a dry, rehydratable thin film culture device comprising a gelling agent.

In another aspect, the present disclosure provides a method for detecting antibiotic-resistant microorganisms. The method can comprise providing a liquid sample suspected of containing antibiotic-resistant microorganisms; providing a dry, thin film culture device; inoculating the culture device with the liquid sample; incubating the inoculated culture device for a period of time; and analyzing the culture device for the presence of antibiotic-resistant microorganisms. The culture device can comprise a nutrient medium, an effective amount of β-lactam antibiotic to select for the growth of antibiotic-resistant microorganisms with the proviso that the β-lactam antibiotic is not aztreonam, an indicator system to indicate the presence of microorganisms, and a gelling agent.

In another aspect, the present disclosure provides a method for detecting and differentiating antibiotic-resistant microorganisms. The method can comprise providing a liquid sample suspected of containing antibiotic-resistant microorganisms; providing a dry, thin film culture device; inoculating the culture device with the liquid sample; incubating the inoculated culture device for a period of time; and analyzing the culture device for the presence of antibiotic-resistant microorganisms. The culture device can comprise a nutrient medium, an effective amount of β-lactam antibiotic to select for the growth of antibiotic-resistant microorganisms with the proviso that the β-lactam antibiotic is not aztreonam, an indicator system to indicate the presence of microorganisms, a second indicator system to indicate the presence of Staphylococcus aureus, and a gelling agent.

In another aspect, the present disclosure provides a method for detecting and differentiating antibiotic-resistant microorganisms. The method can comprise providing a liquid sample suspected of containing antibiotic-resistant microorganisms and a dry, thin film culture device comprising a gelling agent and any one or more of the following ingredients: a nutrient medium, an effective amount of β-lactam antibiotic to select for the growth of antibiotic-resistant microorganisms with the proviso that the β-lactam antibiotic is not aztreonam, and an indicator system to indicate the presence of microorganisms. The method further can comprise adding to the culture device, if not already present, a nutrient medium, an effective amount of β-lactam antibiotic to select for the growth of antibiotic-resistant microorganisms with the proviso that the β-lactam antibiotic is not aztreonam, and an indicator system to indicate the presence of microorganisms. The method further can comprise inoculating the culture device with the liquid sample, incubating the inoculated culture device for a period of time, and analyzing the culture device for the presence of antibiotic-resistant microorganisms.

In another aspect, the present disclosure provides a method for detecting and differentiating antibiotic-resistant microorganisms. The method can comprise providing a liquid sample suspected of containing antibiotic-resistant microorganisms and a dry, thin film culture device comprising a gelling agent and any one or more of the following ingredients: a nutrient medium, an effective amount of β-lactam antibiotic to select for the growth of antibiotic-resistant microorganisms with the proviso that the β-lactam antibiotic is not aztreonam, a first indicator system to indicate the presence of microorganisms, and a second indicator system to detect the presence of S. aureus. The method further can comprise adding to the culture device, if not already present, a nutrient medium, an effective amount of β-lactam antibiotic to select for the growth of antibiotic-resistant microorganisms with the proviso that the β-lactam antibiotic is not aztreonam, and a first indicator system to indicate the presence of microorganisms, and a second indicator system to detect the presence of S. aureus. The method further can comprise inoculating the culture device with the liquid sample, incubating the inoculated culture device for a period of time, and analyzing the culture device for the presence of antibiotic-resistant microorganisms.

### DEFINITIONS

For the purposes of this disclosure,

“liquid sample” refers to an aqueous mixture, including food samples and clinical samples, including those which are homogenized, diluted, and/or suspended in the aqueous mixture, that can contain various microorganisms therein;

“β-lactam antibiotic” refers to any antibiotic that comprises a β-lactam nucleus in its molecular structure. Nonlimiting examples of β-lactam antibiotics include penicillin, cephalosporin, monobactam, carbapenem, and β-lactam-containing derivatives of any of the foregoing;

“powder” refers to particulate material of one or more gelling agents having an average diameter suitable for use in the thin film culture plate device(s) of the present invention, preferably a diameter of about 10-400 microns more preferably a diameter of about 30-90 microns;

“cold-water-soluble powder” refers to a powder that forms a gel in room temperature water (e.g., about 18° C. to 24° C.) when combined with an aqueous test sample;

“non-inhibitory emulsifying agent” refers to an emulsifying agent, preferably a nonionic emulsifying agent, which is suitable to disperse a water-insoluble adhesive in an aqueous environment and does not substantially inhibit the growth of the microorganisms intended to be grown;

“reconstituted medium” refers to a solution and/or gel formed from the reconstitution of a cold-water-soluble powder with water or an aqueous test sample;

“air-permeable” refers to a material which, when substantially exposed at its edges to air, is sufficiently permeable to air in the horizontal direction (i.e., parallel to its top and bottom surfaces) to provide an adequate supply of air to an overlying reconstituted medium in order to support the growth of aerobic microorganisms in the reconstituted medium;

“water-based adhesive composition” refers to an adhesive composition of a water-insoluble adhesive which is dispersed in an aqueous environment by a non-inhibitory; and

“selective agent” refers to any element, compound, or composition that functions to inhibit the growth, and/or facilitate the identification, of microorganisms grown on the culture media device(s) according to the present invention.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a sample suspected of containing “a” microorganism can be interpreted to mean that the liquid can include “one or more” microorganisms.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawing figures listed below, where like structure is referenced by like numerals throughout the several views.

FIG. 1 is a top perspective view, partially in section, of an embodiment of a thin film culture device comprising a spacer.

FIG. 2 is a top view of one embodiment of a self-supporting substrate comprising a grid pattern.

FIG. 3 is a top perspective view, partially in section, of an embodiment of a thin film culture device.

FIG. 4 is a top perspective view, partially in section, of an embodiment of a surface colony counting thin film culture device comprising a spacer and a capture element.

### DETAILED DESCRIPTION

The present disclosure relates to articles and methods for detecting antibiotic-resistant (e.g., β-lactam antibiotic-resistant) microorganisms in a sample. The present disclosure further provides articles and methods to differentiate antibiotic-resistant microorganisms in a sample. In particular, the disclosure relates to the detection of antibiotic-resistant staphylococci. In some embodiments, antibiotic-resistant Staphylococcus aureus can be differentiated from antibiotic-resistant coagulase-negative staphylococci. In contrast to conventional presence/absence tests for antibiotic-resistant microorganisms, the inventive methods provide for the quantitation of viable antibiotic-resistant microorganisms in a sample. The inventive methods further provide for the quantitation of different species and/or groups of antibiotic-resistant microorganisms in a sample.

Conventional agar-based tests for detecting antibiotic-resistant microorganisms require the labor-intensive preparation of the agar plates and, in general, the plates must be used within a relatively short period of time to avoid dehydration and/or loss of potency of the antibiotic. In contrast, the dry, thin-film culture devices of the present disclosure are sample-ready, can be stored for relatively long periods of time, and, optionally, the antibiotic can be added during use, thereby assuring full potency of the antibiotic selection.

Conventional methods for detecting antibiotic-resistant microorganisms typically involve purifying colonies of microorganisms and transferring them to agar plates containing antibiotics to determine whether they can grow in the presence of the antibiotic. In these methods, the presence of antibiotic-resistant microorganisms initially is detected qualitatively (i.e., colonies on a streak-plate or growth in a presence-absence broth culture) and, subsequently, the antibiotic resistance pure colonies of the detected microorganism is characterized (i.e., by generating an antibiogram or by conducting minimal inhibitory concentration (MIC) or minimal bactericidal concentration (MBC) determinations). The conventional methods may include differential tests (e.g., agglutination assays) to enable a presumptive identification of the antibiotic-resistant microorganism. However, the conventional methods do not provide for the direct, quantitative enumeration of the antibiotic-resistant microorganisms in the original sample. Furthermore, conventional methods do not provide for the differential quantitation of mixed populations of microorganisms present in the original sample. The inventive methods of the present disclosure provide for the enumeration of antibiotic resistant microorganisms in the original sample or in a diluted portion of the original sample. The inventive methods of the present disclosure further provide for the differential enumeration of mixed populations of antibiotic microorganisms in a sample.

### Thin Film Culture Devices

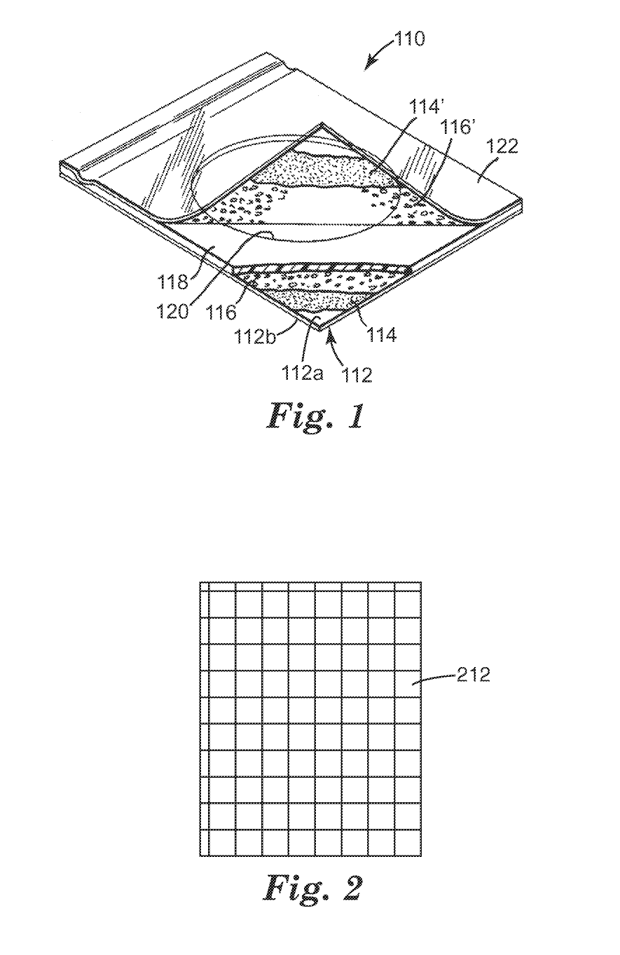

Articles of the present invention include thin film culture devices, such as those disclosed in U.S. Pat. Nos. 4,476,226; 5,089,413, and 5,232,838; which are incorporated herein by reference in their entirety. FIG. 1 illustrates an embodiment of a thin film culture device in accordance with the present invention. The culture device 110 includes a body member comprising a self-supporting water-proof substrate 112 having upper and lower surfaces (112a and 112b, respectively). Substrate 112 can be a relatively stiff film (e.g., polyester, polypropylene or polystyrene), which will not absorb or otherwise be affected by water. The substrate 112 may be either transparent or opaque, depending on whether one wishes to view bacterial colonies through the substrate. To facilitate the counting of bacterial colonies, the substrate 212 can have a grid pattern (e.g., squares) printed thereon, as shown in FIG. 2.

Referring back to FIG. 1, substrate 112 can be coated on its upper surface 112a with a layer of an adhesive 114 which serves to hold the dry gelling agent and/or nutrients in a uniform monolayer for easy hydration. Adhesive 114 should be coated onto substrate 112 in a thickness which is preferably less than the diameter of the particles of the powdered gelling agent and/or nutrients. The object is to apply enough adhesive to adhere the particles to the substrate but not so much that the particles become completely embedded in the adhesive. A uniform monolayer of cold-water-soluble powder 116 is desired with sufficient surface area exposed for hydration. Also shown in FIG. 1 are optional adhesive 114′ and cold-water-soluble powder 116′ layers on cover sheet 122.

In some embodiments, adhesive 114 can comprise a water-based adhesive composition. Preferably, the layer of water-based adhesive 114 is sufficiently transparent when wetted by an aqueous test sample to enable the viewing of the colonies of microorganisms. The water-based adhesive composition can incorporate one or more hydrophilic agents, including nutrients, selective agents, indicators (e.g., enzyme substrates, dyes), or combinations thereof. The specific nutrients and/or selective agents used in the water-based adhesive composition will be apparent to those skilled in the art in view of the present specification depending upon the particular organisms to be grown and/or to be selectively detected (e.g., dyed) or inhibited.

An exemplary useful class of hydrophilic selective agents include dyes that are metabolized by, or otherwise react with, growing microorganisms, and in so doing cause the microbial colonies to be colored or fluoresce for ease of detection and/or quantitation by a technician or by an automated reader. Nonlimiting examples of such dyes include triphenyltetrazolium chloride, p-tolyltetrazolium red, tetrazolium violet, veratryl tetrazolium blue, neutral red, phenol red, chlorophenol red, and 5-bromo-4-chloro-3-indolyl phosphate disodium salt. Particularly preferred dyes in accordance with the present invention include neutral red and 5-bromo-4-chloro-3-indolyl phosphate disodium salt. However, it will be appreciated that other suitable dyes can be used depending on the particular organism(s) to be identified.

In another embodiment of the invention, powder 116 may comprise nutrients but no gelling agent. Gelling agent may be desirable if one desires to visualize and/or isolate discrete bacteria colonies. In many microbiological tests, such as tests for bacteria identification or antibiotic susceptibility, broth media are used, and a viscous gel may not be necessary. In devices for carrying out such tests, the gelling agent may be omitted.

A buffering reagent, such as sodium carbonate, can be employed to provide a medium exhibiting a neutral pH and “Cab-O-Sil M-5” can be employed as a processing aid, as described in U.S. Pat. No. 4,565,783, which is incorporated herein by reference in its entirety. Of course, the particular coating mixture (e.g., nutrients, indicators, and/or gelling agents) used for powder 116 may be adjusted depending upon the type of microorganisms to be grown.

A non-limiting example mixture for powder to support the growth of a variety of staphylococci is as follows:

15 grams gum (1:1 mixture of M150 Guar gum and Xanthan gum (Rhodia, Cranbury, N.J.)

5 grams peptone

2.5 grams yeast extract
1 gram dextrose
0.06 gram sodium carbonate

0.12 gram “Cab-O-Sil M-5” (a fumed silicon dioxide, commercially available from Cabot Corporation)

It is contemplated that articles of the present disclosure can include differential indicators. As used herein, “differential indicator” refers to a reagent added to the medium that will indicate the presence of certain microorganisms and not other microorganisms. Nonlimiting examples of differential indicators include dyes (e.g., stains, pH indicators, redox indicators), enzyme substrates (e.g., chromogenic or fluorogenic substrates for phosphatases, glycosidases, peptidases, nucleases, lipases, and the like), and specific nutrients (e.g., fermentable carbohydrates, amino acids) which, when metabolized by certain microorganisms, produce a detectable reaction (e.g., a pH change associated with a colony).

In some embodiments, one or more differential indicators can be added to the thin film culture device in the water-based composition that is coated onto the substrate. In some embodiments, one or more differential indicators can be added to the liquid sample that is added to the culture device. In some embodiments, one or more differential indicators can be added to the culture device, after hydration of the culture device. An example of a method involving the use of a differential indicator added to the culture device after hydration is the method wherein an article for the detection of thermonuclease is added to the culture device after incubation such as described in U.S. Pat. No. 6,022,682 which is incorporated herein by reference in its entirety.

It is also contemplated within the scope of the invention that powder 116 may optionally include reagents necessary for carrying out certain biochemical tests for microorganism identification. Such reagents (e.g. an enzyme substrate), which undergo a color change in the presence of a particular type of microorganism, may be included in the powder 116 or adhesive 114.

In another embodiment of the invention, powder 116 may comprise a coating that includes a mixture of a gelling agent and a nutrient, a selective agent, and/or an indicator which has been dissolved or suspended in a solution, coated and dried onto substrate 112. In this embodiment, the coating is substantially water-free (i.e., the coating has a water content no greater than about the water content of the dehydrated coating once it has been permitted to equilibrate with the ambient environment).

As depicted in FIG. 1, the body member can include a spacer 118 applied to the upper surface of substrate 112, the spacer 118 comprising a circular aperture 120 cut through the center to expose the powder 116 on substrate 112. The walls of aperture 120 provide a well of predetermined size and shape to confine the medium following hydration. Spacer 118 should be thick enough to form a well of the desired volume, e.g., 1, 2 or 3 milliliters. Closed cell polyethylene foam is a preferred material for spacer 118, but any material which is hydrophobic (non-wetting), inert to microorganisms, and capable of withstanding sterilization may be used. In some embodiments (not shown), the spacer 118 can comprise a plurality of apertures 20 (e.g., 2, 3, 4, 5, 6, 7, 8, 9, 10, 12, 15, or 20 apertures), each of which can be inoculated with a distinct liquid sample.

Spacer 118 can include relatively thick designs, such as those described in U.S. Pat. No. 5,681,712, which is incorporated herein by reference in its entirety. One purpose of the thicker apertured spacer 118 is to locate and protect membranes (e.g. microporous filter membranes) placed in the aperture 120 of the spacer 118 (not shown). Another purpose of the thicker spacer 118 is to reduce or prevent contact by cover sheet 122 with the growing colonies of microorganisms (i.e., provide a “head space” between the growth surface and the cover sheet 122, which can also provide increased aeration for growing colonies of microorganisms).

The thickness of spacer 118 should be sufficient to enclose the liquid volume added to the culture device when the device is inoculated. Depending upon the thickness of the membrane, when used, the spacer can be at least about 0.5 mm thick, about 1 mm thick, about 1.5 mm thick and about 2 mm thick.

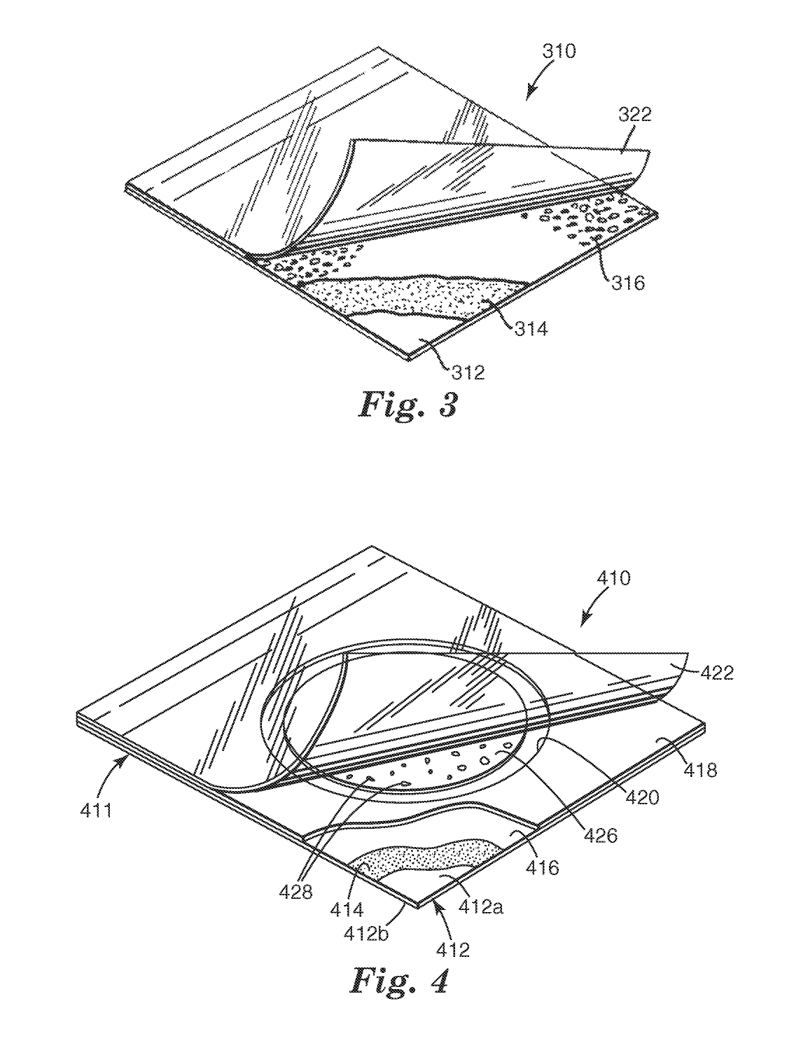

FIG. 3 shows another embodiment of a thin film culture device 310. This embodiment includes substrate 312, adhesive 314, cold-water-soluble powder 316, and cover sheet 322, as described in FIG. 1. In contrast to the culture device 110 of FIG. 1, the device 310 of FIG. 3 does not include a spacer to confine the sample during inoculation. A template, e.g., a weighted ring (not shown), may be applied temporarily to the outside of cover sheet 322, after closing, to confine the sample to a specific region while the cold-water-soluble powder 316 forms a gel. In some embodiments, the device 310 can be inoculated with a plurality (e.g., 2, 3, 4, 5, 6, 7, 8, 9, 10, 12, 15, or 20) of distinct liquid samples, using appropriate spacing and templates to confine the separate samples to distinct portions of the powder 316 of the culture device 310.

FIG. 4 illustrates another embodiment of a thin film culture device 410 in accordance with the present invention. Culture device 410 includes body member 411 comprising self-supporting substrate 412 having upper and lower surfaces 412a and 412b, respectively. Substrate 412 is coated on its upper surface 412a with a layer of adhesive 414. Cold-water-soluble powder 416, comprising one or more gelling agents, is adhered in a thin, relatively uniform layer to the adhesive 414. Once inoculated with an aqueous test sample (not shown), the layer of cold-water-soluble powder 416 quickly hydrates to form a reconstituted medium (not shown), which in turn is capable of growing microorganisms present either in a liquid inoculum or on the surface of a membrane such as a test sample microorganism filter (not shown). Spacer 418 partially covers substrate 412 and the surface of powder 416 and contains aperture 420. In addition, thin film culture device 410 optionally includes cover sheet 422, to cover the reconstituted medium formed after addition of the aqueous test sample. FIG. 4 also shows a capture element 426 and microorganism colonies 428 growing thereon. In the illustrated embodiment, capture element 426 is a microporous membrane through which a liquid sample has been filtered in order to trap any bacteria, if present in the sample, thereon.

Although the embodiments illustrated in FIGS. 1-4 have a cover sheet attached to the device, it is also contemplated within the scope of the invention that the powder-containing embodiments may be uncovered and simply placed in a sterile environment during storage and incubation.

It is possible to use air-permeable membrane layers in the devices of the present invention as described in U.S. Pat. No. 5,232,838. The air permeable layer can be “sandwiched” between the substrate and the cold-water-soluble powder, with an adhesive coating on both sides of the air-permeable membrane layer (not shown).

### Antibiotics

The present disclosure includes articles and methods that utilize antibiotics to select for the growth of antibiotic-resistant microorganisms. Certain microorganisms (e.g., Staphylococcus aureus) isolated from clinical infections are routinely screened for their resistance to certain antibiotics (e.g., β-lactam antibiotics). The choice of antibiotic to use in the screening process can be dependent upon the microorganisms that the operator desires to test for antibiotic resistance.

In some embodiments, the antibiotic can be incorporated into a thin-film culture device during the manufacture of the device. In some embodiments, an antibiotic can be incorporated into an adhesive coating of the thin-film culture device, such as described in Example 1 of U.S. Pat. No. 5,089,413. Additionally, or alternatively, an antibiotic can be incorporated into a mixture which is coated on a substrate during the preparation of a thin-film culture device (such as described in Examples 1 and 2 of U.S. Pat. No. 7,087,401). U.S. Pat. No. 7,087,401, which is incorporated herein by reference in its entirety, describes the incorporation of an antibiotic into an aqueous mixture, which is subsequently coated and dried onto a substrate. In some embodiments, the antibiotic can be added to a mixture of powders (e.g., powdered nutrients and/or a gelling agent) and the mixture can be coated onto an adhesive layer on a substrate using a powder coating process.

In some embodiments, the antibiotic can be added to the thin-film culture device during the use of the device. For example, the antibiotic can be added to the sample before the sample is inoculated into the culture device. Alternatively, a solution containing the antibiotic can be added to the culture device before the sample is inoculated into the device. For example, in one embodiment, the gelling agent of the device is hydrated with an antibiotic-containing solution and the sample is inoculated onto the hydrated gel by, for example, placing a filter containing the sample onto the surface of the hydrated gel. In some embodiments, a concentrated solution of antibiotic is added to the thin-film culture device immediately before the sample is added to the device and the two solutions are mixed during inoculation.

It is contemplated that any antibiotic that is compatible with the thin-film culture device materials can be used to detect and/or enumerate antibiotic-resistant microorganisms according to the present disclosure. “Compatible”, as used herein, means that the antibiotic does not interact with other materials in the thin-film culture device in a way that substantially alters the efficacy of the antibiotic or will adversely affect the ability to grow and detect microorganisms that are resistant to the antibiotic. A preferred group of antibiotics to detect antibiotic-resistant microorganisms is the group of β-lactam antibiotics, which are often the primary type of antibiotic used to treat clinical infections. In some embodiments, a β-lactam antibiotic may be used in the culture device to detect antibiotic-resistant microorganisms. In certain embodiments, the β-lactam antibiotic belongs to the cephalosporin group of β-lactam antibiotics. The cephalosporin group of antibiotics includes “first-generation cephalosporins” (e.g., cefazolin). The cephalosporin group of antibiotics also includes “second-generation cephalosporins” (e.g., cefoxitin and cefuroxime).

In some embodiments, cefazolin can be used in a thin-film culture device according to the present disclosure. The cefazolin can be added to the device, as described above, in an amount that will yield about 1 μg/mL to about 5 μg/mL in the hydrated medium. In some embodiments, the final concentration of cefazolin in the inoculated sample can be about 1 μg/mL, about 3 μg/mL, about 4 μg/mL or about 5 μg/mL.

In some embodiments, cefuroxime can be used in a thin-film culture device according to the present disclosure. The cefuroxime can be added to the device, as described above, in an amount that will yield about 3 μg/mL to about 5 μg/mL in the hydrated medium. In some embodiments, the final concentration of cefuroxime in the inoculated sample can be about 3 μg/mL, about 4 μg/mL or about 5 μg/mL.

In some embodiments, cefoxitin can be used in a thin-film culture device according to the present disclosure. The cefoxitin can be added to the device, as described above, in an amount that will yield about 3 μg/mL to about 5 μg/mL in the hydrated medium. In some embodiments, the final concentration of cefoxitin in the inoculated sample can be about 3 μg/mL, about 4 μg/mL or about 5 μg/mL.

It is contemplated that, within the scope of the present disclosure, the articles or methods may include the use of two or more antibiotics to detect and/or enumerate antibiotic-resistant microorganisms.

### Capture Element

Culture devices of the present disclosure can be used with a capture element to detect antibiotic-resistant microorganisms in a liquid suspension or on a surface. As used herein, “capture element” refers to an article that is used to capture and retain microorganisms that are present in a sample. Preferably, capture elements are dimensioned to allow them to be placed into the thin film culture devices of the present invention and remain in the thin film culture device during the incubation period for a sufficient period to allow for at least one cell division of the target microorganism. Placing the capture element into the culture device can bring the capture element in contact with a gelling agent and/or a nutrient medium, if present, in the culture device, allowing microorganisms to grow and/or proliferate. In some embodiments, the culture device is hydrated (e.g., inoculated with a sterile liquid or an unknown liquid sample) before the capture element is placed into the culture device. In some embodiments, the culture device is hydrated after the capture element is placed into the culture device.

Capture elements can be selected for their suitability with certain types of samples. For example, microporous filters can be used as capture elements to retain microorganisms present in a liquid sample. The liquid sample can be passed through the filter and the microorganisms can be retained thereon. Microorganisms can be retained by, for example, physical entrapment or specific (e.g., antigen-antibody or receptor-ligand interaction) or nonspecific (hydrophobic adsorption) chemical interaction.

Referring to the embodiment shown in FIG. 4, the test sample may comprise a liquid inoculum and/or a capture element 426 such as a microporous filter (e.g., a filter membrane) or a wipe device. Capture element 426 can be constructed from various membranes and films and can be used to capture microorganisms. In some embodiments, capture element 426 can provide a surface on which the colonies of microorganisms can be grown, detected and/or enumerated by the method and devices of the invention. Particularly suitable are known microporous filters which have been commonly used to separate small microorganism populations, such as bacteria from large fluid samples. Such filters are known to be placed on the surface of agar media and incubated to allow counting and evaluation of the filtered microbes. Suitable filters include the HAWG series, e.g., HAWG 04750 type HA filter, available from Millipore Corp. (Marlborough, Mass.) and the Metricel type, e.g., GN-6 Metricel membrane, available from Gelman Corp. (Ann Arbor, Mich.). Other suitable membranes include hydrophilic membranes prepared by providing coatings on various polymers comprising of homo- or copolymers of vinyl alcohol, as described in PCT International Publication No. WO 92/07899. A vinyl alcohol coated microporous polypropylene prepared by the method described in Example 5 of the International Publication is a preferred microorganism filter in the present invention.

Films of the microorganism filters described above are generally relatively thin, about 0.01-2 mm thick and preferably 0.05-1.0 mm thick, and may be provided in any desired 2-dimensional shape, e.g., as rectangles, as discs (including partial discs) and the like.

Microorganisms are separated by such filters with varying efficiency depending upon the sizes of the pores in the membranes. Bacteria are readily separated and yeasts and molds will also be separated by such filters. Filtration is carried out by conventional methods using funnels and discs of suitable sizes. Discs are preferably handled aseptically with tweezers. Discs may be made by the user from commercially available materials or are provided in aseptic packages as separate entities or as parts of kits of the invention.

Wipe devices can be used as capture elements with the culture devices of the present disclosure. As used herein, a “wipe device” is an article that is configured for contacting a surface to obtain a sample of microorganisms disposed thereon. Wipe devices can include porous, nonwoven materials. Nonlimiting examples of wipe materials include paper (e.g., filter paper, cellulosic membrane filters), synthetic nonwovens (e.g., nylon or polyester nonwovens), polymeric or ceramic membranes (e.g., polycarbonate membranes, zirconia membranes), and microstructured films (e.g., microchannel-containing films such as those described in U.S. Pat. No. 7,223,364, which is incorporated herein by reference in its entirety). In some embodiments, the microchannel-containing films comprise through-holes that allow the passage of liquid (and solutes or small particles) from one major surface of the film to the other major surface. Wipe devices can include chemicals (e.g., surfactants), to improve wettability, or reagents (e.g., differential stains). Wipe devices in general comprise chemicals in an amount that will not substantially inhibit the growth of antibiotic resistant microorganisms under the inoculation and incubation conditions described herein. In some embodiments, the capture elements are substantially transparent or become substantially transparent when wet, allowing for the visualization of a differential reaction, such as hemolysis, through the capture element.

Suitable capture elements include a particle, or a plurality of particles. The capture elements include a means for coupling the capture element to microorganisms. Nonlimiting examples of particles include microspheres, microbeads, and the like. Such particles can be resin particles, for example, agarose, latex, polystyrene, nylon, polyacrylamide, cellulose, polysaccharide, or a combination thereof, or inorganic particles, for example, silica, aluminum oxide, or a combination thereof. Such particles can be magnetic, paramagnetic, superparamagnetic, or non-magnetic. Such particles can be colloidal in size, for example about 100 nm to about 10 microns (μm). Nonlimiting examples of such particles include superparamagnetic polymer particles sold under the trade names DYNABEADS (Invitrogen, Inc., Carlsbad, Calif.) and BIO-ADEMBEADS (Ademtech, Pessac, France). Particle capture elements may be incorporated into other structures, such as a microporous membrane.

There are a variety of means for coupling capture element (e.g., a particle) to a microorganism. In some embodiments, the means for coupling the capture element to the microorganism can include surface molecules or properties that promote nonspecific adsorption. For example, at least a portion of the capture element can have molecules on its surface that, under the proper conditions (e.g., high pH or low pH), become positively- or negatively-charged and nonspecifically adsorb to complementary-charged molecules associated with the surface of a microorganism.

Additionally, or alternatively, at least a portion of the capture element (e.g., a polystyrene particle) can have a hydrophobic surface which nonspecifically adsorbs to hydrophobic molecules associated with the surface of a microorganism. In some embodiments, the means for coupling a capture element to a microorganism may comprise a molecule that specifically binds to a microorganism through a receptor-ligand interaction. Such specific receptor-ligand interactions are well known in the art and include interactions between, for example, antibodies and their corresponding antigens, lectins and their corresponding carbohydrate binding partner, bacteriophage proteins and their corresponding phage receptors, and the like. It should be understood that the means for coupling a particle to a microorganism can also be used in conjunction with film or non-woven (e.g., filter) capture elements, as well as the particulate capture elements.

### Samples

Antibiotic-resistant species of interest can be analyzed in a test sample that may be derived from any source, such as a physiological fluid, e.g., blood, saliva, ocular lens fluid, synovial fluid, cerebral spinal fluid, pus, sweat, exudate, urine, mucus, feces, lactation milk, or the like. Further, the test sample may be derived from a body site, e.g., wound, skin, nares, scalp, nails, etc.

Samples of particular interest include mucus-containing samples, such as nasal samples (from, e.g., anterior nares, nasopharyngeal cavity, nasal cavities, anterior nasal vestibule, etc.), as well as samples from the outer ear, middle ear, mouth, rectum, vagina, or other similar tissue. Examples of specific mucosal tissues include buccal, gingival, nasal, ocular, tracheal, bronchial, gastrointestinal, rectal, urethral, ureteral, vaginal, cervical, and uterine mucosal membranes.

Besides physiological fluids, other test samples may include other liquids as well as solid(s) dissolved in a liquid medium. Samples of interest may include process streams, water, soil, plants or other vegetation, air, surfaces (e.g., contaminated surfaces, floors, walls, instruments, bedding), and the like. Samples can also include cultured cells.

Various patient sampling techniques for the detection of microbes, such as S. aureus, on surfaces are known. Such sampling techniques are suitable for the methods of the present invention as well. For example, it is common to obtain a sample from wiping the nares of a patient. A particularly preferred sampling technique includes the subject's (e.g., patient's) anterior nares swabbed with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for one or more complete revolutions along the nares' mucosal surface.

A wide variety of swabs or other sample collection devices are commercially available, for example, from Puritan Medical Products Co. LLC, Guilford, Me., under the trade designation PURE-WRAPS or from Copan Diagnostics, Inc. Corona, Calif., under the trade designation ESWAB, or from microRheologics, Sid., Brescia, IT, under the trade designation FLOCKEDSWAB. A sample collection means such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like.

The sample collection device (e.g., swab) can then be cultured directly, analyzed directly, or extracted (e.g., by washing, elution by vortexing) with an appropriate solution. Such extraction (i.e., elution) solutions typically include water and can optionally include a buffer and at least one surfactant. An example of an elution buffer includes, for example, phosphate buffered saline (PBS), which can be used in combination, for example, with TWEEN 20 or PLURONIC L64. The test sample (e.g., liquid) may be subjected to treatment prior to further analysis. This includes concentration, precipitation, filtration, centrifugation, dialysis, dilution, inactivation of natural components, addition of reagents, chemical treatment, etc.

Other sample collection devices, also referred to as “capture elements”, can be used to collect samples from a surface or from liquid stream. In some embodiments, a capture element can be used to wipe a surface to collect a representative sample of microorganisms from the surface. Subsequently, the capture element can be transferred into a culture device, where it remains during incubation and growth of the microorganisms. In some embodiments, a capture element can be used to filter microorganisms out of a liquid sample. After the filtration step, the capture element can be transferred into a culture device, where it remains during incubation and growth of the microorganisms.

### Methods for Detecting and Differentiating Methicillin-Resistant Microorganisms

Thin film devices can be used in methods to detect and differentiate antibiotic-resistant (e.g., methicillin-resistant) microorganisms. The devices can be inoculated and the dry nutrients and/or gelling agent can be hydrated by several different procedures. In some embodiments a liquid sample can be inoculated into the thin film device, essentially forming a “pour plate”. In some embodiments, an aqueous liquid (e.g., water, a buffer, a nutrient medium), which is free of the target flora and which may be sterile, can be added to the thin film device and the gel is allowed to solidify. After the gel solidifies, the coversheet may be opened and the gel can be brought into contact with a surface (e.g., a wall, a floor, an instrument, skin, a mucous membrane) to inoculate the device. The coversheet subsequently can be closed for incubation. In some embodiments, the aqueous solution is added to the device, as described above and a capture device (e.g., a membrane filter or a wipe) can be placed subsequently into the thin film device, thereby inoculating the device. Alternatively, moisture associated with the capture element can be used to dissolve the nutrients and gelling agent in the thin-film. In some embodiments, an aqueous solution can be added to the device to dissolve the nutrients and gelling agent after a capture element has been placed in the device. In some embodiments, a sample solution can be distributed (e.g., deposited, spread or streaked) into the device prior to adding the aqueous solution to dissolve the nutrients and gelling agent. A weighted plate or a specially-designed spreader can be placed on top of the cover sheet to spread the sample completely.

The thin film devices can further be used in methods to enumerate antibiotic-resistant microorganisms. After inoculation, the culture device is then incubated for a predetermined period of time. Bacterial colonies growing in or on the medium or capture element can be counted through the cover film.

At least one hydrophilic agent such as, for example, a nutrient, an indicator, or a selective agent (e.g. an antibiotic) can be added to the culture device with the sample at the time of inoculation. The at least one hydrophilic agent, which is preferably sterile, can be dissolved, suspended, or diluted into the liquid sample before adding the sample to the culture device. In some embodiments, the at least one hydrophilic agent can be added separately to the culture device (e.g., as a powder, a dried film, a coating on a substrate, or a solution) immediately before or immediately after the liquid sample and, optionally, can be mixed with the sample (e.g., with a pipette tip) in the culture device before closing and/or incubating the culture device.

Hydrophilic agents (e.g., a nutrient, an antibiotic, and indicator) can be added to an inoculated/hydrated culture device by contacting a liquid, semi-solid solution or a dehydrated article (e.g., coated, dried substrate) with the hydrated nutrient medium in the culture device after the culture device has been inoculated. The dehydrated article may be a partially-dehydrated article. The substrate can comprise a plastic film, a microporous membrane, a cellulosic material, or a nonwoven material.

Differential indicators, if present, can be used to distinguish between and provide a differential count of different groups or species of microorganisms. For example, enzyme substrates can be used to differentiate between colonies containing staphylococci and colonies containing Bacillus species or other microorganisms. U.S. Pat. No. 5,837,482, which is incorporated herein by reference in its entirety, describes an indicator system using an indolyl-glucopyranoside enzyme substrate to detect non-staphylococcal microorganisms and Baird-Parker differential reagents (e.g., Egg Yolk-Tellurite suspension) to detect staphylococcal microorganisms. U.S. Pat. No. 5,635,367, which is incorporated herein by reference in its entirety, describes an indicator system using an indolyl-glucopyranoside enzyme substrate to detect non-staphylococcal microorganisms and an indolyl-phosphate enzyme substrate to detect staphylococcal microorganisms.

Colonies can be picked from the culture device to perform differential tests. Colonies can be tested individually or they can be grouped, or “pooled”, for differential testing. Colonies can be “pooled”, for example, by picking two or more colonies from the device, mixing them together, and performing a differential test simultaneously on microorganisms from the two or more colonies. The differential tests can include, for example, staining tests (e.g. Gram stain, spore stain, immunochemical staining), enzymatic (e.g., a DNase test, a TNase test), surface receptor recognition tests (e.g., coagulase test or clumping factor test), genetic tests (e.g., amplification tests, such as PCR and rtPCR; nucleic acid sequencing; or hybridization assays (e.g., FISH assays)), immunoassay tests (e.g., ELISA, immunodiffusion, immunochromatography), or biochemical tests (e.g., coagulase test, catalase test, carbohydrate fermentation (e.g., mannitol fermentation), lipid analysis)

### Kits of the Invention

Kits provided by the present invention include two or more parts. One part includes a thin film culture plate device described herein. A second part of each kit may be selected from the group of accessory articles consisting of a membrane filter, a pipette, a spreader, a glove, a sample acquisition device, a capture element, a sample-suspending medium, a reagent, and any two or more of the foregoing accessory articles.

Membrane filters should be of a shape and size that is suitable for fitting into the aperture of the spacer of the culture plate device of the kit. Filters of different kinds can be provided with a kit, or multiple kits can contain various filters. The filters are optional and, preferably, provided in aseptic condition such as a polyethylene coated paper package which has been sterilized by gamma irradiation, ethylene oxide or other sterilization. Alternatively the filters may be nonsterile units which are to be sterilized by the user.

Suitable pipettes and spreaders can be made from, for example, plastic or glass. The pipettes and spreaders can be provided in a pre-sterilized condition or can be provided in a nonsterile condition. The pipettes and spreaders can be disposable after a single use or can be resterilized for multiple uses. “Pipettes”, as used herein include volumetric pipettes with at least one gradation mark corresponding to a known volume and pipette tips, which can be used with a volumetric pipetting device. The kit can contain a package of hydrophilic agents. The hydrophilic agents are preferably contained in a sterile package for example a foil package such as those conventionally used in the pharmaceutical industry. An example of such a package is used for NITRO-BID Ointment (Marlon Laboratories, Inc., Kansas City, Mo.).

The nutrients and/or selective agents included in the kits may be incorporated into the adhesive and/or powder compositions, as discussed above. The selection of the hydrophilic agents useful and necessary in the kits depends upon the microorganism to be evaluated. Another criterion for selection of components of a kit will be short and long term chemical compatibility of the hydrophilic agents.

The invention will be further illustrated by reference to the following non-limiting Examples. All parts and percentages are expressed as parts by weight unless otherwise indicated. Unless specified otherwise, all reagents were obtained from Sigma Chemical Company (St. Louis, Mo.).

### EXAMPLES

### Example 1

### Detection and Enumeration of Staphylococcus Species in a Thin-Film Culture Device Containing Cefoxitin or Oxacillin

### Preparation of Reagents and Media

PETRIFILM Aerobic Count Plates were obtained from 3M Company (St. Paul, Minn.). DIFCO dehydrated tryptic soy agar (TSA) was obtained from BD Diagnostics (Sparks, Md.). Defibrinated sheep blood was obtained from Remel, Inc. (Lenexa, Kans.).

Phosphate-buffered saline (PBS), pH 7.5, was prepared from a 10× concentrate (OmniPur 6505 buffered saline; EMD Chemicals, Inc.; Gibbstown, N.J.) and was sterilized by autoclaving. Antibiotic stock solutions were prepared in deionized water and were sterilized by passage through a 0.22 μm filter.

Sheep blood agar containing cefoxitin (SBA-Cf) was prepared to compare the recovery of antibiotic resistant microorganisms on agar media to the recovery of the antibiotic-resistant microorganisms in a thin-film culture device. Twenty grams of dehydrated TSA was mixed with 475 mL of deionized water and autoclaved. The molten agar was tempered at 50° C. Twenty-five milliliters of defibrinated sheep blood was added to the agar (to yield a final concentration of 5% sheep blood) and swirled to mix. Cefoxitin stock solution (0.5 mL of 4 mg/mL) was added to the agar and swirled to mix. The agar was poured into sterile petri dishes and the agar was allowed to solidify at room temperature. The plates were inverted and stored at 4° C.

PBS diluent solutions were prepared for diluting the bacterial cultures. The diluent solutions contained cefoxitin at 3 μg/mL, 4 μg/mL, and 5 μg/mL, respectively, or oxacillin at 4 μg/mL, 5 μg/mL, and 6 μg/mL, respectively. The diluent solutions were used within eight hours after they were prepared. PBS without antibiotic was the diluent used for the PETRIFILM and SBA control plates.

### Preparation of Bacterial Suspensions

Eight strains of staphylococcal microorganisms were tested. Methicillin-sensitive Staphylococcus epidermidis (strain 96), methicillin-resistant Staphylococcus epidermidis (strains 471 and 472) and methicillin-resistant Staphylococcus aureus (strains 560 and 907) were obtained from human clinical isolates. Methicillin-sensitive Staphylococcus aureus (strains ATCC25923 and ATCC27664) and methicillin-sensitive Staphylococcus epidermidis ATCC 12228 were obtained from the American Type Culture Collection (Manassas, Va.).

### Inoculation, Incubation and Colony Counts

Bacterial strains were inoculated into tryptic soy broth and were incubated overnight at 37° C. The bacterial suspensions were diluted in each of the PBS diluents described above. One-milliliter samples were plated onto the PETRIFILM plates according to the manufacturer's instructions. The cefoxitin-containing blood agar plates were inoculated by spreading 0.1 milliliters of the bacterial suspension over the surface of the plate using a sterile spreader. All plates were incubated at 35° C. and the colonies on each plate were counted at 24±2 hours and 48±4 hours. The colony counts (CFUs) are shown in Tables 1 and 2. The data indicate that methicillin-resistant S. aureus and S. epidermidis can be detected in both type of plating media (agar and PETRIFILM plates) after 24 and 48 hours of incubation. The data show that strain 560 is sensitive to cefoxitin and resistant to oxacillin.

TABLE 1
Colony-forming units (CFUs) observed at 24 hours on PETRIFILM plates and blood agar plates
containing cefoxitin or oxacillin.

| Strain # | 12228 | 96 | 471 | 472 | 25923 | 27664 | 560 | 907 |
|---|---|---|---|---|---|---|---|---|
| Dil. Factor | 2 × 10⁻⁶ | 1 × 10⁻⁶ | 2 × 10⁻⁶ | 1 × 10⁻⁶ | 1 × 10⁻⁶ | 1 × 10⁻⁶ | 2 × 10⁻⁶ | 2 × 10⁻⁶ |
| PF-C | 184 | 94 | 178 | 130 | 41 | 167 | 108 | 155 |
| PF-Cf3 | 0 | 0 | 143 | 90 | 0 | 0 | 102 | 170 |
| PF-Cf4 | 0 | 0 | 130 | 112 | 0 | 0 | 100 | 158 |
| PF-Cf5 | 0 | 0 | 150 | 88 | 0 | 0 | 109 | 152 |
| PF-Ox4 | 0 | 0 | 68 | 29 | 0 | 0 | 0 | 166 |
| PF-Ox5 | 0 | 0 | 48 | 14 | 0 | 0 | 0 | 154 |
| PF-Ox6 | 0 | 0 | 30 | 14 | 0 | 0 | 0 | 152 |
| SBA-Cf | 0 | 0 | 152 | 103 | 0 | 0 | 85 | 140 |
| SBA-C | 164 | 160 | 168 | 88 | 36 | 100 | 66 | 134 |

The dilution factor (Dil. Factor) shown in the table is the final dilution factor, which includes the difference in the volume of sample inoculated into the respective plate types.
PF-C = PETRIFILM plate control,
PF-Cf(n) = Petrifilm plates with cefoxitin at the designated number of micrograms/mL,
PF-Ox(n) = Petrifilm plates with oxacillin at the designated number of micrograms/mL,
SBA-Cf = sheep blood agar containing cefoxitin,
SBA-C = sheep blood agar control

TABLE 2
Colony-forming units (CFUs) observed at 48 hours on PETRIFILM plates and blood agar plates
containing cefoxitin or oxacillin.

| Strain # | 12228 | 96 | 471 | 472 | 25923 | 27664 | 560 | 907 |
|---|---|---|---|---|---|---|---|---|
| Dil. Factor | 2 × 10⁻⁶ | 1 × 10⁻⁶ | 2 × 10⁻⁶ | 1 × 10⁻⁶ | 1 × 10⁻⁶ | 1 × 10⁻⁶ | 2 × 10⁻⁶ | 2 × 10⁻⁶ |
| PF-C | 194 | 100 | 180 | 134 | 41 | 168 | 108 | 158 |
| PF-Cf3 | 0 | 0 | 144 | 99 | 0 | 0 | 102 | 171 |
| PF-Cf4 | 0 | 0 | 136 | 128 | 0 | 0 | 100 | 158 |
| PF-Cf5 | 0 | 0 | 154 | 107 | 0 | 0 | 109 | 154 |
| PF-Ox4 | 0 | 0 | 140 | 76 | 0 | 0 | 0 | 166 |
| PF-Ox5 | 0 | 0 | 131 | 66 | 0 | 0 | 0 | 154 |
| PF-Ox6 | 0 | 0 | 115 | 62 | 0 | 0 | 0 | 152 |
| SBA-Cf | 0 | 0 | 152 | 103 | 0 | 0 | 85 | 140 |
| SBA-C | 164 | 161 | 169 | 89 | 36 | 100 | 67 | 134 |

The dilution factor shown in the table is the final dilution factor, which includes the volume of sample inoculated into the respective plate types.
Abbreviations are the same as those reported in Table 1.

The present invention has now been described with reference to several specific embodiments foreseen by the inventor for which enabling descriptions are available. Insubstantial modifications of the invention, including modifications not presently foreseen, may nonetheless constitute equivalents thereto. Thus, the scope of the present invention should not be limited by the details and structures described herein, but rather solely by the following claims, and equivalents thereto.

1. An article for detecting or enumerating antibiotic-resistant microorganisms, the article comprising:
an effective amount of a β-lactam antibiotic to select for the growth of antibiotic-resistant staphylococcal microorganisms, with the proviso that the β-lactam antibiotic is not aztreonam;
a nutrient medium;
an indicator system to indicate the presence of microorganisms; and
a dry, rehydratable thin film culture device comprising a gelling agent.

2. An article for differentially enumerating antibiotic-resistant microorganisms, the article comprising:
an effective amount of a β-lactam antibiotic to select for the growth of antibiotic-resistant staphylococcal microorganisms, with the proviso that the β-lactam antibiotic is not aztreonam;
a nutrient medium;
a first indicator system to indicate the presence of microorganisms;
a second indicator system to indicate the presence of Staphylococcus aureus; and
a dry, rehydratable thin film culture device comprising a gelling agent.

3. The article of claim 1, wherein the antibiotic comprises methicillin, oxacillin, or a cephalosporin antibiotic.

4-5. (canceled)

6. The article of claim 1, wherein the antibiotic comprises cephoxitin, wherein the concentration of cefoxitin in the rehydrated nutrient medium of the culture device is about 0.5 μg/mL to about 4 μg/mL.

7-10. (canceled)

11. The article of claim 1, further comprising potassium tellurite.

12. The article of claim 1, further comprising lithium chloride.

13. The article of claim 1, further comprising a second antibiotic.

14. The article of claim 13, wherein the second antibiotic is not a β-lactam antibiotic.

15. The article of claim 1 wherein the thin film culture devices comprises a surface colony counting thin film culture device.

16. A method for detecting and antibiotic-resistant microorganisms, comprising:
providing a liquid sample suspected of containing antibiotic-resistant microorganisms; and a dry, thin film culture device comprising a nutrient medium, an effective amount of β-lactam antibiotic to select for the growth of antibiotic-resistant microorganisms with the proviso that the β-lactam antibiotic is not aztreonam an indicator system to indicate the presence of microorganisms, and a gelling agent;
inoculating the culture device with the liquid sample;
incubating the inoculated culture device for a period of time; and
analyzing the culture device for the presence of antibiotic-resistant microorganisms.

17. A method for detecting and differentiating antibiotic-resistant microorganisms, comprising:
providing a liquid sample suspected of containing antibiotic-resistant microorganisms; and a dry, thin film culture device comprising a nutrient medium, an effective amount of β-lactam antibiotic to select for the growth of antibiotic-resistant microorganisms with the proviso that the β-lactam antibiotic is not aztreonam, a first indicator system to indicate the presence of microorganisms, a second indicator system to indicate the presence of Staphylococcus aureus, and a gelling agent;
inoculating the culture device with the liquid sample;
incubating the inoculated culture device for a period of time; and
analyzing the culture device for the presence of antibiotic-resistant microorganisms.

18. A method for detecting and differentiating antibiotic-resistant microorganisms, comprising:
providing a liquid sample suspected of containing antibiotic-resistant microorganisms; and a dry, thin film culture device comprising a gelling agent and any one or more of the following ingredients: a nutrient medium, an effective amount of β-lactam antibiotic to select for the growth of antibiotic-resistant microorganisms with the proviso that the β-lactam antibiotic is not aztreonam, or an indicator system to indicate the presence of microorganisms;
adding to the culture device, if not already present, a nutrient medium, an effective amount of β-lactam antibiotic to select for the growth of antibiotic-resistant microorganisms with the proviso that the β-lactam antibiotic is not aztreonam, and an indicator system to indicate the presence of microorganisms;
inoculating the culture device with the liquid sample;
incubating the inoculated culture device for a period of time; and
analyzing the culture device for the presence of antibiotic-resistant microorganisms.

19. A method for detecting and differentiating antibiotic-resistant microorganisms, comprising:
providing a liquid sample suspected of containing antibiotic-resistant microorganisms; and a dry, thin film culture device comprising a gelling agent and any one or more of the following ingredients: a nutrient medium, an effective amount of β-lactam antibiotic to select for the growth of antibiotic-resistant microorganisms with the proviso that the β-lactam antibiotic is not aztreonam, a first indicator system to indicate the presence of microorganisms, or a second indicator system to indicate the presence of Staphylococcus aureus;
adding to the culture device, if not already present, a nutrient medium, an effective amount of β-lactam antibiotic to select for the growth of antibiotic-resistant microorganisms with the proviso that the β-lactam antibiotic is not aztreonam, a first indicator system to indicate the presence of microorganisms, and a second indicator system to indicate the presence of Staphylococcus aureus;
inoculating the culture device with the liquid sample;
incubating the inoculated culture device for a period of time; and
analyzing the culture device for the presence of antibiotic-resistant microorganisms.

20-21. (canceled)

22. The method of claim 16, further comprising the steps of i) providing a capture element and ii) contacting the gelling agent with the capture element.

23. The method of claim 22, wherein the gelling agent is hydrated before the capture element is contacted with the gelling agent.

24. (canceled)

25. The method of claim 16 wherein analyzing the medium for the presence of antibiotic-resistant microorganisms comprises enumerating colonies.

26. The method of claim 16, further comprising the step of performing a differential test on microorganisms from one or more colonies.

27. The method of claim 26, wherein microorganisms from one or more colonies are removed from the culture device to perform the differential test.

28. The method of claim 27, wherein microorganisms from two or more colonies are removed from the culture device and mixed together to perform the differential test.

29. The method of claim 26, wherein the differential test comprises a biochemical test.

30-34. (canceled)

35. The method of claim 16, wherein a hydrophilic agent is contacted with the hydrated nutrient medium in the culture device after the culture device has been inoculated.

36-38. (canceled)
